(19) United States
(12) Patent Application Publication
(10) Pub. No.: US 2010/0292564 A1
(43) Pub. Date: Nov. 18, 2010

(54) System and Method For Magnetic-Nanoparticle, Hyperthermia Cancer Therapy

(76) Inventor: Padraig J. Cantillon Murphy, Cambridge, MA (US)

Correspondence Address: QUARLES & BRADY LLP, 411 E. WISCONSIN AVENUE, SUITE 2040, MILWAUKEE, WI, 53202-4497, US

(21) Appl. No.: 12/781,902

(22) Filed: May 18, 2010

Related U.S. Application Data
(60) Provisional application No. 61/179,256, filed on May 18, 2009.

(51) Int. Cl. A61B 5/055

(52) U.S. Cl. 600/411

(57) ABSTRACT

A system and method is provided for performing and monitoring a magnetic nanoparticle hyperthermia process on a subject having received a dose of a magnetic nanoparticle configured to bind to tissue within a target area of the subject. A low-field MRI system is utilized having a static magnetic field and a radio frequency (RF) system configured to receive MRI data from the target area. A hyperthermia system is coupled to the low-field MRI system and configured to generate a hyperthermia excitation field configured to cause the magnetic nanoparticle to rotate at a lag with respect to a magnetic field experienced by the magnetic nanoparticle to cause a temperature increase in the target area. The MRI system is configured to acquire medical imaging data from the target area and reconstruct images therefrom indicating at least one of a spatial distribution of the nanoparticle in and a temperature of the target area.

### CROSS-REFERENCE TO RELATED APPLICATIONS

This application is based on and claims the benefit of U.S. Provisional Application Ser. No. 61/179,256 filed on May 18, 2009, and entitled “A Method and Apparatus For Combing Magnetic Nanoparticle Hyperthermia with MRI for Cancer Therapy”, which is incorporated herein by reference.

### STATEMENT REGARDING FEDERALLY SPONSORED RESEARCH

This invention was made with United States government support under National Institute of Environmental Health Sciences Grant No. R01EB007942.

### BACKGROUND OF THE INVENTION

The present invention relates generally to the use of magnetic nanoparticle hyperthermia (MPH) and, more particularly, to the use of a magnetic resonance imaging (MRI) system and MPH to treat cancer tumors.

Recent years have seen the development of an entire field of research dedicated to the treatment of cancer patients based on the localized heating effects of magnetic nanoparticles in vivo. Treatment falls under one of two categories, hyperthermia and thermoablation.

In hyperthermia, the target area is subjected to a local temperature in the range of 42° C. to 45° C. for periods of up to a few hours. This temperature range is destructive to cancerous cells without harming healthy cells. The end result is usually not sufficient to eradicate all the cancerous cells and is coupled with other techniques, such as irradiation or chemotherapy. Thermoablation aims at creating in vivo temperatures in excess of 50° C. in the tumor region and exposure time is limited to just minutes. Although this approach would appear preferred, there are concerns regarding the physiological effects of such a rapid, localized heating effect. To date, magnetic particles that have been used are either injected in situ and/or are designed to bind selectively to cancerous cells. The majority of investigations use superparamagnetic magnetite (Fe₃O₄) or maghemite (γFe₃O₄) in water based suspensions since these are well metabolized. Creating in vivo concentration to allow for sufficient heating is a major challenge due to heat conduction away from the target area as well as blood perfusion around the tumor. A second significant challenge is the undesired heating associated with eddy currents in surrounding healthy tissue. It has been found that subjects had a sensation of warmth but were able to withstand the treatment for more than an hour when the product of the RF hyperthermia field amplitude and frequency did not exceed 4.85×10⁸ A/m/s.

Magnetic resonance imaging (MRI) uses the nuclear magnetic resonance (NMR) phenomenon to produce images. When a substance such as human tissue is subjected to a uniform magnetic field (“main,” or polarizing, magnetic field, B₀), the individual magnetic moments of the nuclei in the tissue attempt to align with this polarizing field, but precess about it in random order at their characteristic Larmor frequency. If the substance, or tissue, is subjected to a magnetic field (excitation field, B₁) that is in the x-y plane and that is near the Larmor frequency, the net aligned moment, M_{Z}, referred to as longitudinal magnetization, may be rotated, or “tipped,” into the x-y plane to produce a net transverse magnetic moment, M_{xy}, referred to as transverse magnetization. A signal is emitted by the excited nuclei or “spins,” after the excitation field, B₁, is terminated, and this signal may be received and processed to form an image.

When utilizing these “MR” signals to produce images, magnetic field gradients (Gₓ, G_{y}, and G_{z}) are employed for spatial encoding. Typically, the region to be imaged is scanned by a sequence of measurement cycles in which these gradients vary according to the particular localization method being used. The resulting set of received MR signals are digitized and processed to reconstruct the image using one of many well known reconstruction techniques.

The notion of a combining MRI with RF ablation techniques has been attempted numerous times to varying levels of success. Other imaging modalities have also been employed for simultaneous imaging and/or thermometric analysis coupled with hyperthermia. These efforts include laser heat deposition in conjunction with MRI and ultrasonic hyperthermia coupled with MRI. However, in each of these instances, the techniques are limited by the nature of ablation and/or the need to coordinate multiple imaging and therapy modalities. The latter constraint can be quite cumbersome, particularly, when the imaging modality is MRI, which necessarily creates an operating environment that is generally quite spatially confined, due to the size constraints of MRI bores, and operationally complex, due to the high static magnetic field and RF pulses required for the imaging acquisitions.

Therefore it would be desirable to have a system and method that combines imaging and therapeutic capabilities, while overcoming the difficulties and drawbacks presented by the above-listed systems and method that rely on the combination of multiple imaging and therapy systems and/or potential negative physiological effects of aggressive therapies, such as ablation.

### SUMMARY OF THE INVENTION

The present invention overcomes the aforementioned drawbacks by providing a system and method that utilizes a low-field MRI environment to perform both magnetic resonance imaging (MRI) and magnetic nanoparticle hyperthermia (MPH). That is, the present invention provides a system and method for the treatment of cancer tumors with a combination of magnetic nanoparticle hyperthermia (MPH) and real-time magnetic resonance imaging (MRI).

A system is provided for performing and monitoring a magnetic nanoparticle hyperthermia process on a subject having received a dose of a magnetic nanoparticle configured to bind to tissue within a target area of the subject. A low-field MRI system is utilized having a static magnetic field and a radio frequency (RF) system configured to receive MRI data from the target area. A hyperthermia system is coupled to the low-field MRI system and configured to generate a hyperthermia excitation field configured to cause the magnetic nanoparticle to rotate at a lag with respect to a magnetic field experienced by the magnetic nanoparticle to cause a temperature increase in the target area. The MRI system is configured to acquire medical imaging data from the target area and reconstruct images therefrom indicating at least one of a spatial distribution of the nanoparticle in and a temperature of the target area.

Also, method is provided for performing a magnetic nanoparticle hyperthermia (MPH) process on a subject having received a dose of a magnetic nanoparticle configured to bind to tissue within a target area of the subject and monitoring the MPH process using a magnetic resonance imaging (MRI) system. The method includes acquiring, with the MRI system having a static magnetic field, images of the target area to determine a desired binding of the magnetic nanoparticle to tissue within the target area. The method also includes producing, with the MRI system, a hyperthermia excitation field configured to cause the magnetic moment associated with the magnetic nanoparticle to rotate at an angular displacement or lag with respect to a magnetic field experienced by the magnetic nanoparticle giving rise to a magnetic torque density. This magnetic torque density can cause a temperature increase in the target area. The method further includes acquiring medical imaging data from the target area during the temperature increase in the target area and reconstructing images of the target area from the medical imaging data to provide real-time feedback indicating at least one of a spatial distribution of the nanoparticle in and a temperature of the target area.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a low-field magnetic resonance imaging (MRI) system configured to also perform a magnetic nanoparticle hyperthermia (MPH) process in accordance with the present invention;

FIG. 2 is a block diagram of a transceiver system that forms part of the MRI system of FIG. 1 in accordance with the present invention;

FIG. 3 is a flow chart setting forth the steps of a method for performing a combined MRI and MPH process in accordance with the present invention;

FIG. 4 is a graph illustrating a dependence of the nanoparticle time constant, τ, on nanoparticle radius, γ;

FIG. 4 is a graph illustrating the Langevin function as a function of the DC MRI filed strength, B₀, in Tesla for various nanoparticle magnetic core radii;

FIGS. 5a-5c are graphs illustrating an effect of varying magnetic nanoparticle concentration on temperature changes, ΔT, versus particle radius, according to three fraction solid magnetic volume values φ=0.01, 0.005, and 0.002; and

FIGS. 6a-6c are graphs illustrating an effect of varying magnetic nanoparticle concentration on temperature changes, ΔT, versus particle radius, according to three μ₀Hₑ values μ₀H=10 mT, 20 mT and 30 mT.

### DETAILED DESCRIPTION OF THE INVENTION

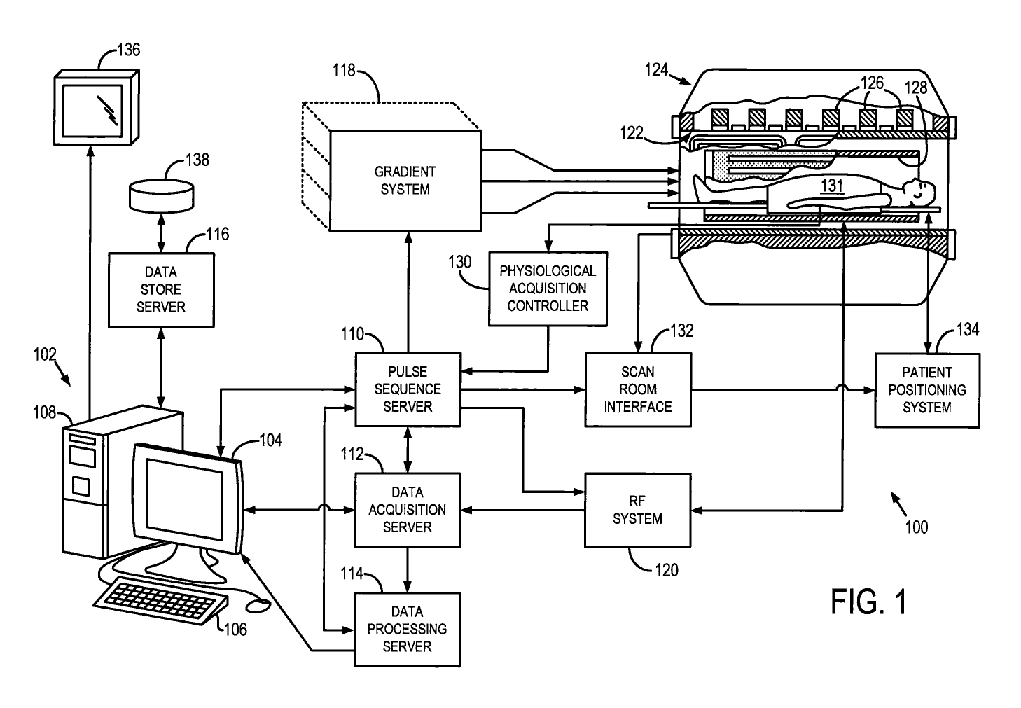

Referring particularly to FIG. 1, the present invention includes a low-field, magnetic resonance imaging (MRI) system 100. As defined herein, “low-field” or “low, static-magnetic field” refers to a class of MRI systems that employ a static magnetic field that is substantially reduced from the most common, MRI system used clinically for imaging purposes, such as MRI systems employing a 1.5 T or 3.0 T static magnetic filed. For example, a “low-field” or “low, static-magnetic field” MRI system, may utilize a static magnetic field of 0.1 T, 0.2 T, 0.35 T, 0.5 T, 0.7 T, or the like. As will be described, the low-field MRI system 100, as illustrated may have an “open bore” or “open magnet” design to further facilitate access to the subject; however, other low-field MRI systems having traditional “closed bores” are also contemplated for use with the present invention.

Since MRI image resolution improves with field strength, B₀, most clinical systems operate at either 1.5 T or 3 T. However, as will be described, at sufficiently high field strengths, magnetic nanoparticles become saturated, that is, their magnetic moment is essentially “locked” in parallel with the DC field, B₀. Under these conditions, the nanoparticles are unable to rotate and generate heating effects in the presence of an RF hyperthermia field. Therefore, the present invention uses a low-field MRI system operating at conventional field strengths and superparamagnetic nanoparticles to perform hyperthermia treatment within am MRI system configured to provide real-time imaging of the nanoparticles and hyperthermia treatment. Superparamagnetic MRI contrast agents such as Feridex are generally delivered in very low concentrations into the bloodstream by direct injection while nanoparticles used in magnetic nanoparticle hyperthermia (MPH) are usually injected into the tumor site and do not enter the vasculature. Feridex is a registered trademark of Advanced Magnetics, Inc. of Cambridge, Mass.

The low-field MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system. The workstation 102 is coupled to four servers: a pulse sequence server 110; a data acquisition server 112; a data processing server 114, and a data store server 116. The workstation 102 and each server 110, 112, 114 and 116 are connected to communicate with each other.

The pulse sequence server 110 functions in response to instructions downloaded from the workstation 102 to operate a gradient system 118 and a radiofrequency (RF) system 120. For imaging purposes, gradient waveforms necessary to perform the prescribed scan are produced and applied to the gradient system 118, which excites gradient coils in an assembly 122 to produce the magnetic field gradients (Gₓ, G_{y}, and G_{z}) used for position encoding MR signals. The gradient coil assembly 122 forms part of a magnet assembly 124 that includes a polarizing magnet 126 and a whole-body or local RF coil 128.

The RF excitation waveforms are applied to the RF coil 128, or a separate local coil (not shown in FIG. 1), by the RF system 120 to perform the prescribed magnetic resonance pulse sequence to acquire MR image data. Specifically, responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays (not shown in FIG. 1).

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} Eqn. (1);

and the phase of the received MR signal may also be determined:

$$φ = {tan}^{- 1} ( \frac{Q}{I} ) . \qquad Eqn . ( 2 )$$

In accordance with the present invention, the RF system 120 is also designed to drive an RF heating coil 131. As will be described, it is contemplated that the subject may be receive a dose of magnetic nanoparticles designed to selectively bind to cancerous lesion(s) in a target site by means of smart chemical surfactants. Thereafter, an RF hyperthermia field is achieved within the target area by controlling the RF system 120 to drive the RF heating coil 131 to affect a desired therapeutic effect and/or cancer necrosis. For example, heating can typically be conducted for periods of less than 10 minutes at a time, which has been shown, in the presence of temperature increases of 4 to 6° C. above the body ambient, to affect a desired therapeutic effect and/or cancer necrosis. Specifically, local increases in temperature are predicted in regions of high nanoparticle concentration due to the rotation of the nanoparticle's magnetic moment in response to the RF field produced by the RF heating coil 131. Using the low-field MRI system 100, during the treatment, real-time, non-invasive tracking of concentration and temperature can be performed. Further, objective evidence of nanoparticle migration away from the target site can likewise be tracked using the low-field MRI system.

During imaging, the pulse sequence server 110 also optionally receives patient data from a physiological acquisition controller 130. The controller 130 receives signals from a number of different sensors connected to the patient, such as electrocardiograph (ECG) signals from electrodes, or respiratory signals from a bellows or other respiratory monitoring device, or temperature monitors.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

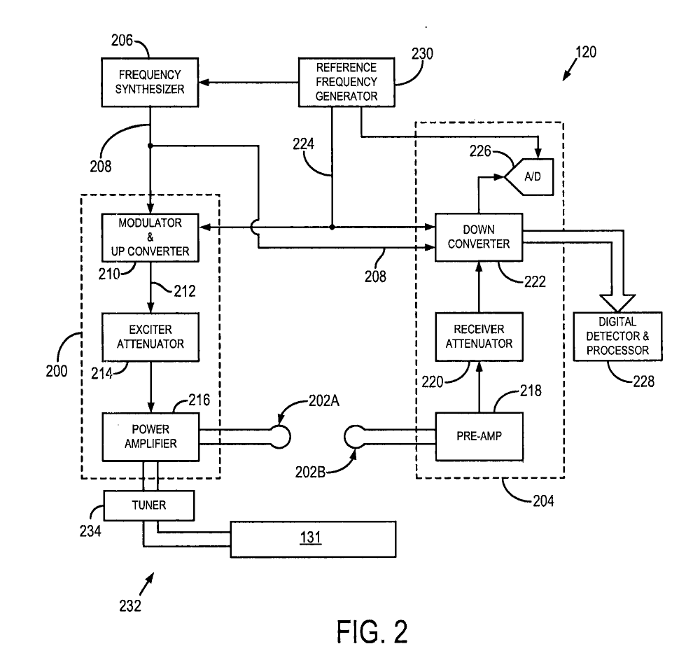

As shown in FIG. 1, the RF system 120 may be connected to a whole-body or local RF coil 128, or as shown in FIG. 2, a transmitter section of the RF system 120 may connect to one RF coil 202A and its receiver section may connect to a separate RF receive coil 202B.

Referring particularly to FIG. 2, the RF system 120 includes a transmitter that produces a prescribed RF excitation field for imaging. The base, or carrier, frequency of this RF excitation field is produced under control of a frequency synthesizer 206 that receives a set of digital signals from the pulse sequence server 110, of FIG. 1. These digital signals indicate the frequency and phase of the RF carrier signal produced at an output 208. The RF carrier is applied to a modulator and up converter 210 where its amplitude is modulated in response to a signal, R(t), also received from the pulse sequence server 110. The signal, R(t), defines the envelope of the RF excitation pulse to be produced and is produced by sequentially reading out a series of stored digital values. These stored digital values may be changed to enable any desired RF pulse envelope to be produced.

The magnitude of the RF excitation pulse produced at output 212 is attenuated by an exciter attenuator circuit 214 that receives a digital command from the pulse sequence server 110, of FIG. 1. The attenuated RF excitation pulses are applied to a power amplifier 216, which drives the imaging RF coil array 202A.

Referring still to FIG. 2, the signal produced by the subject is picked up by the coil array 202B and applied to a pre-amplifier 218 that amplifies the signal by an amount determined by a digital attenuation signal received from the pulse sequence server 110, of FIG. 1. The received signal is at or around the Larmor frequency, and this high frequency signal is pass through a receiver attenuator 220 and, generally, down-converted in a two step process by a down converter 222, which first mixes the detected signal with the carrier signal on line 208 and then mixes the resulting difference signal with a reference signal on line 224. The down converted MR signal is applied to the input of an analog-to-digital (A/D) converter 222 that samples and digitizes the analog signal and applies it to a digital detector and signal processor 228 that produces 16-bit in-phase (I) values and 16-bit quadrature (Q) values corresponding to the received signal. The resulting stream of digitized I and Q values of the received signal are output to the data acquisition server 112, of FIG. 1. The reference signal, as well as the sampling signal applied to the A/D converter 226, are produced by a reference frequency generator 230.

In addition to operating the RF system 120 to perform an MR imaging process, the RF system 102 is designed to drive a hyperthermia system 232. The hyperthermia system 232 is coupled to the power amplifier 216 of the RF system 120 includes an RF heating coil 131 that, as described with respect to FIG. 1, is configured to be arranged proximate a target area to drive a magnetic nanoparticle hyperthermia (MPH) process. As will be described, the RF heating coil could be operated in a multiplexed or time-sharing mode with the MR imaging process either using a separate RF heating coil or using the same coil as that for the MR imaging.

Though in the illustrated configuration, the hyperthermia system 232 is shown as including a separate RF heating coil 131, it is contemplated that the imaging an heating coils may be combined. For example, the combined RF coil has an adequate power rating for the hyperthermia treatment but can multiplex between imaging and hyperthermia settings and is controlled and monitored from the MRI console. Of course, as illustrated, the RF hyperthermia coil may be an independent coil that is meshed with the RF MRI coil to avoid shielding effects. Both coils are then operated in parallel from the MRI console in a similar manner to that employed for multi-channel imaging applications, for example, 32-channel and 64-channel neuroimaging coils. The RF hyperthermia coil may operate in the 100-400 MHz range in order to interact with nanoparticle time constants on the order of μs. This requires significant power amplification for successful operation of a rotating field with amplitude between 10 and 30 mT. The rotating field frequency and amplitude is tuned to match the T of the nanoparticle such that energy transfer and, hence, local temperature increase is maximized at the tumor site, as will be described below. For this reason, a tuning mechanism 234 is incorporated into the invention such that the RF hyperthermia coil can be tuned to maximize heating effects depending on the nanoparticle radius.

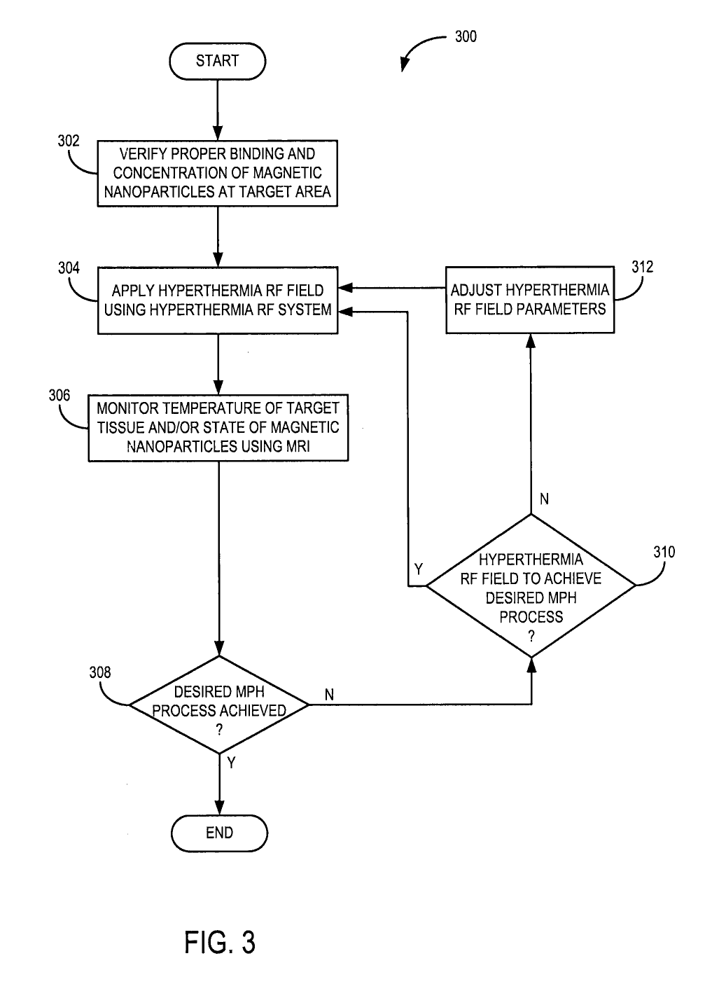

In particular, turning to FIG. 3, a flow chart is provided to illustrate exemplary steps of a process for performing a combined MRI and MPH process, for example, using a system such as described above with respect to FIGS. 1 and 2. The process 300 begins after the administration of magnetic nanoparticles designed to selectively bind to target tissues, such as the cancerous lesion(s), by means of smart chemical surfactants that are highly selective in their binding mechanism. In particular, the process 300 begins by performing an MRI data acquisition designed to verify proper binding and concentration of the magnetic nanoparticles at the tumor site, as indicated at process block 302.

After verify proper binding and concentration of the magnetic nanoparticles at the tumor site at process block 302, the MRI system is utilized to drive the above-described hyperthermia system to increase the temperature of the target tissue by applying RF energy specifically designed to influence the spins of the magnetic nanoparticles, as indicated at process block 304. In keeping with best medical practices relating to MPH, the heating is generally performed for periods of, for example, less than 10 minutes at a time. Significant therapeutic effect and cancer necrosis has been shown in the presence of temperature increases of 4 to 6 degrees Celsius above the body's ambient temperature, which, as will be described below, is achievable using the low-field MRI system and hyperthermia system described above, even when performing real-time imaging to monitor the hyperthermia process. Specifically, local increases in temperature are predicted in regions of high magnetic nanoparticle concentration due to the rotation of the magnetic nanoparticle's magnetic moment in response to the hyperthermia RF field. Rather than an ohmic heating effect, as is generally the used in conventional hyperthermia treatment, the present invention localizes temperature increases to the target site where the nanoparticles bond to the tumor, thereby significantly reducing heating in nearby healthy tissue. As will be explained in detail below, the temperature increase over an appropriate range of magnetic fluid concentrations and nanoparticle radii have been examined and it has been shown that therapeutically significant heating can take place, even in low-field MRI systems where magnetic fluid saturation is not significant, with careful application of a rotating or sinusoidal magnetic field.

As stated above, while the low-field MRI system and hyperthermia system are performing the hyperthermia process, it is possible to also perform real-time imaging using the low-field MRI system to monitor the hyperthermia process, as indicated at process block 306. For example, a variety of thermometric methods may be employed, such as evaluating how T₁ relaxation time of the MRI changes with temperature. This is not the case with current hyperthermia treatment techniques where success is invariably evaluated after the patient is removed from the RF hyperthermia coil. Real-time MRI allows the radiologist to continuously evaluate progress over the treatment as well as providing continuous, non-invasive monitoring of in vivo temperature. Because MPH is highly suited to low blood perfusion tissue such as breast and prostate, the method is highly suitable for cancer affecting these tissues.

As the hyperthermia process is being monitored using the low-field MRI system at process block 306, at decision block 308, a check is made using the real-time MRI data do determine whether the desired MPH process has been achieved. If the desired heating over the desired duration has not been achieved, at decision block 310, a check is made using the real-time MRI data to determine whether the hyperthermia RF field, if continued using the current operational parameters, is on pace to achieved the desired MPH process in the desired time period. If not, the hyperthermia RF field is adjusted at process block 312 and the hyperthermia RF field is applied using the new operational parameters at process block 304. If the hyperthermia RF field is sufficient to achieve the desired MPH process in the desired time, the hyperthermia RF field is applied at process block 304 and monitoring of the temperature of the target tissue is continued at process block 306 until the desired MPH process is complete and the process ends.

The two important parameters are utilized to substantially optimize energy transfer from the RF hyperthermia coil to the target tumor site. In particular, these parameters are the magnetic field heating frequency, f, and the nanoparticle time constant, τ. In the presence of RF sinusoidal or rotating magnetic fields, magnetic nanoparticles will act to realign their magnetic moment with the applied rotating field. The realignment is characterized by the nanoparticle's time constant, τ. Typical values of τ for 5-10 nm diameter nanoparticles lie in the μs range. In fact, τ is a combination of two distinct contributions, known as the Brownian (τ_{B}) and Néel (τ_{N}) time constants as given by:

$$\frac{1}{τ} = \frac{1}{{τ}_{B}} + \frac{1}{{τ}_{N}} . \qquad Eqn . ( 3 )$$

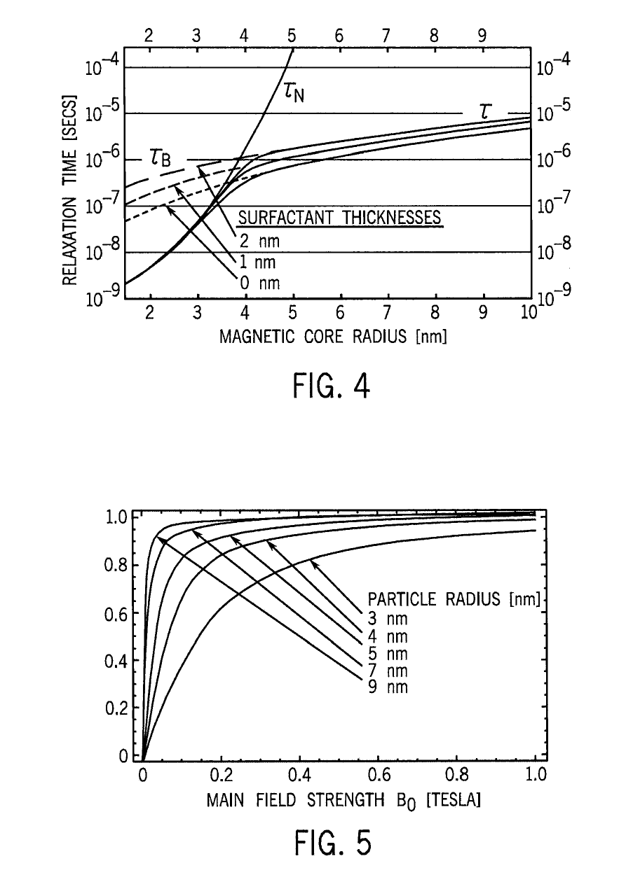

FIG. 4 is a graph illustrating the dependence of the nanoparticle time constant, τ, on the nanoparticle radius, r. Specifically, for smaller nanoparticles, the Néel relaxation dominates; while as nanoparticle radius increases, Brownian relaxation is the dominant mechanism. This is an important distinction because, while Brownian relaxation entails rotation of the entire nanoparticle in the presence of an RF field, Néel relaxation can take place even if the nanoparticle's physical rotation is constrained, for example, by adherence to a tumor. In the case of Néel relaxation, it is only the magnetic moment of the particle that rotates and this relaxation mechanism is dominant for smaller nanoparticles.

In the presence of a sinusoidal or rotating RF hyperthermia field, the magnetic nanoparticle will rotate such that the instantaneous RF field and the magnetic moment of the nanoparticle are maintained in parallel. As the magnetic field frequency is increased, the nanoparticle's magnetic moment begins to lag the applied rotating magnetic field at a constant angle for a given frequency, Ω=2πf, in rad/s. This lag is characterized by a nonzero magnetic torque density in the magnetic nanoparticle suspension, with which is associated an increase in the bulk suspension temperature. Significant heating occurs as Ωτ approaches unity. Present invention recognizes this phenomenon and exploits the rise in the magnetic fluid's temperature in the MRI environment, which is characterized by a large DC magnetic field, B₀, perpendicular to the RF hyperthermia field. In the presence of B₀, energy transfer can be controlled with careful selection of the system parameters, such RF hyperthermia field amplitude, frequency, and phase.

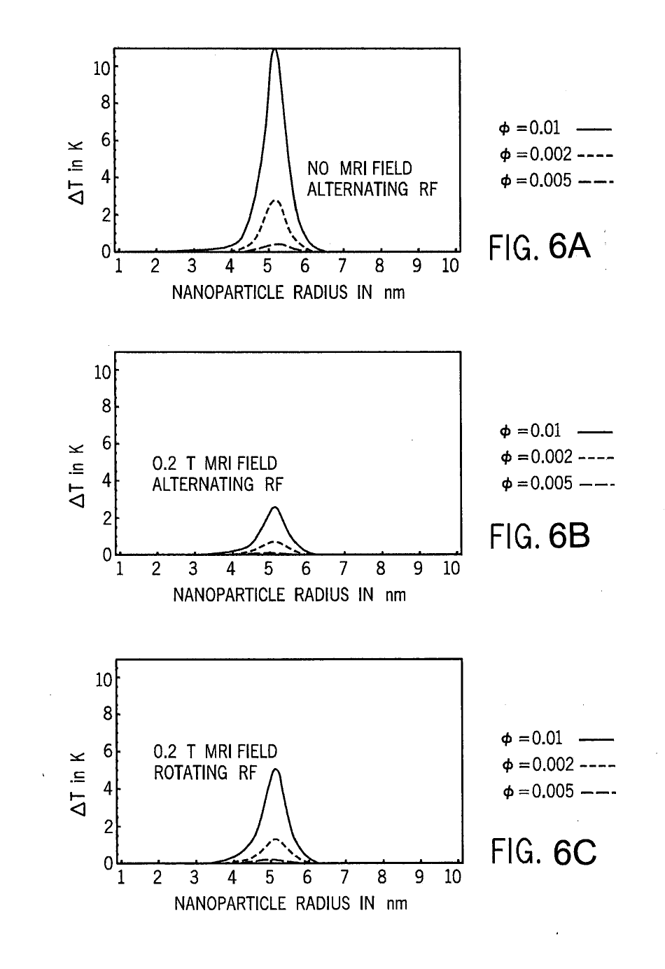

FIG. 5 is a graph illustrating the Langevin function as a function of the DC MRI filed strength, B₀, in Tesla for various nanoparticle magnetic core radii. Magnetic nanoparticle saturation in a fluid suspension due to magnetic fields is determined by the Langevin function, given by Eqn. (3) and plotted in FIG. 5, where M_{eq} is the equilibrium magnetization of the suspension in the direction of the saturation magnetization, M_{S′}, both in units of A/m. The Langevin parameter, α, depends on the permeability of free space which has a value of μ₀=4π×10⁻⁷N·A⁻², the magnetic core particle volume, Vₚ, the single domain magnetization of the particle, M_{d}, which has a value of 446×10³ A/m for magnetite, a common constituent in such particles, the magnitude of the local magnetic field intensity in the suspension, ¦H¦, and the thermal energy density given by the product of the Boltzmann constant, k, and the absolute temperature, T in Kelvin. The saturation magnetization, M_{S}, is related to M_{d} such that M_{S}=φM_{d}, where φ is the fraction solid magnetic volume in the fluid suspension.

$$\begin{matrix}{M}_{eq} = {M}_{s} {L}_{( α ) =} {M}_{s} ( coth ( α ) - 1 / α ) . & Eqn . ( 4 ) \\ α = \frac{{μ}_{0} {V}_{p} {M}_{d} \vert H \vert}{kT} . & Eqn . ( 5 )\end{matrix}$$

At low fields, the Langevin function, L(α) approaches zero indicating that the nanoparticles thermal kinetic energy dominates the magnetic energy. For increasing magnetic fields, such as those typical in clinical, closed-bore, imaging MRI system having, for example, a static magnetic field of 1.5 T and 3 T, the magnetic energy density, given by the numerator of Eqn. (5), is dominant and the magnetic nanoparticles are effectively saturated or “locked” in parallel with the large DC field characteristic of such MRI systems. However, at lower field strengths, such as those used in open or low-field MRI, such as 0.2 T and 0.35 T, the nanoparticles are not entirely saturated, as indicated in FIG. 54, such that in the presence of a sinusoidal or rotating magnetic field applied perpendicular to B₀, they are at least partially free to rotate.

The rotation of the magnetic nanoparticles in the presence of either a sinusoidal or rotating field is governed by three equations. These are Shliomis' relaxation equation (Eqn. (6) below), the conservation of linear momentum, (Eqn. (7) below), and the conservation of angular momentum (Eqn. (8) below):

$$\begin{matrix}\frac{\partial M}{\partial t} + ( v \cdot ∇ ) M - ω \times M = - \frac{1}{τ} ( M - {M}_{eq} ) ; & Eqn . ( 6 ) \\ 0 = ρ g + {μ}_{0} M \cdot ∇ H - ∇ p + ( n + ϛ ) {∇}^{2} v + 2 ϛ ( ∇ \times ω ) ; & Eqn . ( 7 ) \\ 0 = {μ}_{0} M \times H + 2 ϛ ( ∇ \times v - 2 ω ) ; & Eqn . ( 8 )\end{matrix}$$

where M is the instantaneous suspension magnetization in A/m, v is the fluid flow velocity in m/s, v̅ is the vector differential operator, ω the fluid suspension spin velocity in rad/s, ρ is the fluid suspension density in kg/m³, g is the gravitational acceleration constant in m/s², p is the fluid suspension pressure in Pa, η is the fluid suspension dynamic shear viscosity in N·s/m², and ζ is the fluid suspension vortex viscosity in vortex viscosity which is approximately given by

$$\frac{3}{2} ϕ \cdot η$$

for dilute suspensions in units of N·s/m².

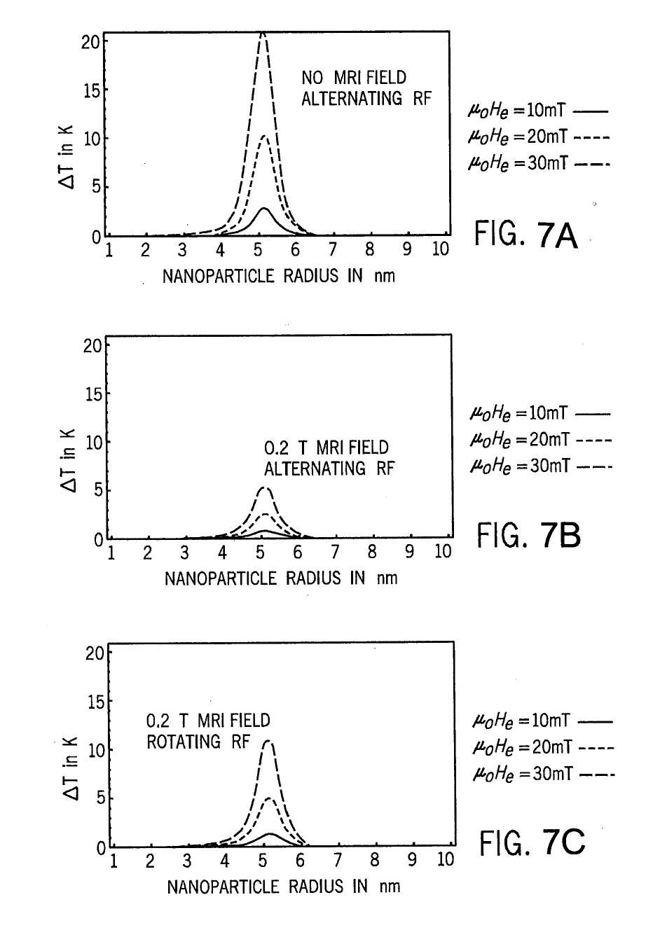

This complex equation set is not readily solvable except by numerical methods. However, a convenient case study of a planar channel can be analyzed such that analytical solutions are possible for demonstration of heating effects in the fluid suspension. This is the case considered in the analytical results that will be shown in FIGS. 6a-c and 7a-c.

Again, a rotating or sinusoidal magnetic field is applied perpendicular to the large B₀ field characteristic of the MRI system, which causes nanoparticle rotation, characterized by the spin-velocity, ω. The suspension saturation is governed by the B₀ field for the case where the B₀ is significantly larger than the sinusoidal or rotating field amplitude, less than 30 mT in this example. The effect of the sinusoidal or rotating magnetic field is the misalignment of the nanoparticle magnetic moments with the applied field when Ωτ approaches unity, as already noted.

In addition to the governing equations given by Eqn. 6 through Eqn. 8, heating in a magnetic fluid suspension is governed by the First Law of Thermodynamics as follow:

dU=∂Q+∂w Eqn. (9)

where the differential internal heat density, dU, is given by the contributions due to the differential energy input, ∂Q, and the differential work output, ∂W.

For an adiabatic process, approximated by low-perfusion tissue, such as the breast or prostate tissue, ∂Q is approximately zero and for a suspension of magnetic nanoparticles, ∂w is given by dU. The resulting increase in internal energy density for cyclic variations in the nanoparticle magnetization, |M|=M and the magnetic field, H, is given by:

$$Δ U \approx ∮ \partial W \approx {μ}_{0} ∮ M \cdot d H . \qquad Eqn . ( 10 )$$

To illustrate how this result can be used to predict the local temperature increase in low-perfusion tissue, small spherical tumors of radius, R, are assumed and the resulting steady-state increase in local temperature is given by:

$$Δ T = \frac{{R}^{2} f 〈 Δ U 〉}{3 λ} ; \qquad Eqn . ( 11 )$$

where the [symbol] brackets symbolize a time-averaged internal energy density while the tissue's heat conductivity, λ, of tissue with an approximate value of 0.64 W/K/cm.

FIGS. 6a-6c provide graphs illustrating the effect of varying magnetic nanoparticle concentration on the increase in temperature ΔT versus particle radius, according to three fraction solid magnetic volume values φ=0.01, 0.005, and 0.002. In FIG. 6a, no MRI, static magnetic field is applied and the increase in temperature is due to an alternating-sinusoidal magnetic field of 10 mT. In FIG. 6b, a 0.2 T MRI field is utilized as well as an alternating-sinusoidal magnetic field of 10 mT. Finally, in FIG. 6c, a 0.2 T MRI field is used along with a rotating field of 10 mT. In each of these three plots, the alternating or rotating magnetic field is applied in the plane perpendicular to the MRI field.

Turning to FIGS. 7a-7c, a plurality of graphs are provided that show the effect of varying magnetic nanoparticle concentration on the increase in temperature ΔT versus particle radius, according to three μ₀Hₑ values μ₀Hₑ=10 mT, 20 mT and 30 mT. In FIG. 7a, no MRI, static magnetic field is applied and the increase in temperature is due to an alternating-sinusoidal magnetic field and nanoparticle concentration, φ=0.005. In FIG. 7b, a 0.2 T MRI field is applied and an alternating-sinusoidal magnetic field to a nanoparticle concentration of φ=0.005. Finally, in FIG. 7c, a 0.2 T MRI field is applied along with a rotating magnetic field to a nanoparticle concentration of φ=0.005.

The results of FIGS. 6a-6c and 7a-7c indicate the expected steady-state temperature increase in a 1 cm radius tumor when either a sinusoidal alternating or rotating magnetic field is applied perpendicular to the MRI field. As is evident from FIGS. 6a-6c and 7a-7c, the steady-state temperature rise is a maximum at a particle radius of approximately 5 nm for a rotating RF hyperthermia field frequency of 300 kHz in the absence of a static MRI field. As one would expect, this is where the product of Ωτ approaches unity. Therefore, it should be apparent that careful selection of the hyperthermia field amplitude, frequency, and phase can result in maximized energy transfer to the nanoparticle suspension at the tumor site.

After the addition of the MRI's B₀ field, which characterizes MRI system, the most striking result is the large decrease in ΔT. This decrease is greater as B₀ increases. The presence of the B₀ field causes the fluid to approach saturation far more rapidly than occurs in the presence of the rotating field. Saturation impedes nanoparticle rotation, and hence, heat generation, since one can imagine that as a larger fraction of the nanoparticles remain saturated, their magnetic moments remain collinear with the B₀ field and do not respond to the sinusoidal field excitation orthogonal to the B₀ field. This result is supported by comparing FIGS. 6a and 6b as well as 7a and 7b. Since the DC B₀ field itself does not add or subtract heat from the fluid, the dramatic change in heating can only be accounted for by the partial magnetic saturation which occurs in its presence. It will again be apparent that careful selection of the rotating or sinusoidal field frequency, Ω, is required in order to maximize the heating associated with any given nanoparticle radius, characterized by the time constant, τ. For this reason, a tuning mechanism is incorporated into the invention such that the RF hyperthermia coil can be tuned to maximize heating effects depending on the nanoparticle radius.

Adding a second orthogonal field component to the previous case of an alternating-sinusoidal field has the effect of creating a rotating field if the two components are temporally displaced by a quarter cycle. The results for a rotating field, as illustrated in FIGS. 6c and 7c, in addition to the static MRI B₀ field show an approximate factor of two increase in ΔT compared to the case of a purely alternating-sinusoidal field albeit at the cost of a second RF power amplifier. The increase in temperature is now therapeutically significant (>4° C.) and therefore, indicates that a combined MRI/hyperthermia system is a tenable treatment mechanism for cancer therapy in low-perfusion tissue types, such as the breast and prostate.

While combined conventional hyperthermia and MRI systems have been proposed and constructed previously, such as the mini-annular phased array developed at the National Institutes of Health, which operating a time-sharing arrangement between image acquisition and hyperthermia treatment and, more recently, Gellermann et al. have reported “reasonable trends and correlations” using a combined 1.5 T MRI platform with a hybrid hyperthermia coil inserted into the magnet bore, these systems have all relied on ohmic heating. That is, previous attempts to combine hyperthermia and MRI systems generally ignored the use of nanoparticles for the reasons listed above with respect to the problems using nanoparticles with traditional imaging MRI system having static magnetic fields at 1.5 T and above.

Other imaging modalities have also been employed for simultaneous imaging and/or thermometric analysis coupled with hyperthermia. These include laser heat deposition in conjunction with MRI and ultrasonic hyperthermia coupled with MRI. However, in each of these cases, the imaging modality (MRI) and the hyperthermia source are not integrated but rather consist of two distinct and independent systems. The attraction of a combined RF hyperthermia system coupled with low-field MRI is that the heating source can be made to interface seamlessly with the MRI system since the MRI platform already has a built-in RF source, used for excitation of the hydrogen protons during imaging. Although the current MRI RF field, usually referred to as the B₁ field, is not suitable for hyperthermia treatment since the amplitude of the MRI RF field is too small (˜μT) and the frequency invariant, the same interface may be used for the RF hyperthermia field source presented in the present invention either by a practice of timesharing or by parallel connections.

Magnetic particle hyperthermia is under investigation by numerous groups, although a thorough review indicates that none of these investigators have considered integrating MPH with MRI. In vivo rodent trials have taken place in Japan which reported “71% tumor suppression” due to MPH alone. Another recent development is the Magnetic Drug Targeting (MDT) developed by Siemens Medical Solutions where an external focused magnetic field source is brought to bear on injected magnetic fluid for the application of targeted delivery of the magnetic particles. However, the device does not appear to afford simultaneous imaging of the target. Much recent work has focused on the functionalization of magnetic nanoparticles allowing for specific binding to in vivo tumors. In addition, Babincova et al. have proposed a single functionalized nanoparticle which combines cancer chemotherapy and hyperthermia based on iron oxide magnetic nanoparticles functionalized with cisplatin. However, these systems are not focused on simultaneously using nanoparticles for MPH and MR imaging and do not address the numerous technical hurdles overcome by the present invention to facilitate combined MPH and MRI using nanoparticles.

Therefore, the present invention provides a combined MRI/hyperthermia system that includes an MRI system utilizing a low static magnetic field strength. The combined MRI/hyperthermia system also includes an RF hyperthermia coil, a power amplifier, and a control system that is embedded into an operator console of the MRI system, such that control of the RF hyperthermia coil is similar to control of the B₁ RF field. To achieve the desired RF hyperthermia using the MRI system utilizing a low static magnetic field strength, selectively binding magnetic nanoparticles are employed that are designed to act as both an MRI contrast agent for imaging purposes and a hyperthermia treatment mechanism due the nanoparticles' localized temperature increase in the presence of the RF hyperthermia field.

In accordance with another aspect of the invention, a method is provided that includes, after the administration of magnetic nanoparticles into the target site that are designed to selectively bind to cancerous lesion(s) by means of smart chemical surfactants, verifying the binding and concentration of the nanoparticles at the target site using a low-filed MRI system. Thereafter, an RF hyperthermia field is applied to affect a desired therapeutic effect and/or cancer necrosis. Local increases in temperature are predicted in regions of high nanoparticle concentration due to the rotation of the nanoparticle's magnetic moment in response to the RF field. Rather than an ohmic heating effect, as is generally the case in conventional hyperthermia treatment, the technique localizes temperature increases to the target site where the nanoparticles are bond to the tumor, thereby significantly reducing heating in nearby healthy tissue. During the treatment, the MRI system is utilized for real-time, non-invasive tracking of concentration and temperature, as well for evidence of nanoparticle migration away from the tumor site.

The present invention has been described in terms of one or more preferred embodiments, and it should be appreciated that many equivalents, alternatives, variations, and modifications, aside from those expressly stated, are possible and within the scope of the invention.

1. A system for performing and monitoring a magnetic nanoparticle hyperthermia process on a subject having received a dose of a magnetic nanoparticle configured to bind to tissue within a target area of the subject, the system comprising:
a low-field magnetic resonance imaging (MRI) system having a static magnetic field configured to receive a subject arranged therein and a radio frequency (RF) system configured to receive MRI data from a target area in the subject;
a hyperthermia system coupled to the low-field MRI system and configured to generate a hyperthermia excitation field configured to cause a magnetic nanoparticle administered to the subject to rotate at a lag with respect to a magnetic field experienced by the magnetic nanoparticle to cause a temperature increase in the target area; and
wherein the MRI system is configured to acquire medical imaging data from the target area and reconstruct images therefrom indicating at least one of a spatial distribution of the nanoparticle in and a temperature of the target area.

2. The system of claim 1 wherein the hyperthermia system is configured to generate the hyperthermia excitation field to be substantially orthogonal to the static magnetic field of the MRI system.

3. The system of claim 1 wherein the hyperthermia system is configured to generate the hyperthermia excitation field that is at least one of a rotating and a sinusoidal magnetic field applied substantially perpendicular to the static magnetic field.

4. The system of claim 1 wherein the hyperthermia system is configured to generate the hyperthermia excitation field such that the hyperthermia excitation field and a magnetic moment of the magnetic nanoparticle are maintained substantially in a non-parallel alignment.

5. The system of claim 1 wherein the magnetic nanoparticle is configured to operate as a contrast agent during the acquisition of the medical imaging data.

6. The system of claim 1 wherein the hyperthermia system includes a tuner configured to tune at least one of a frequency and an amplitude of the hyperthermia excitation field to substantially match the a time constant of the magnetic nanoparticle.

7. The system of claim 1 wherein the hyperthermia system is further configured to generate another hyperthermia excitation field to create an alternating magnetic field substantially transverse to the static magnetic field of the MRI system.

8. The system of claim 7 wherein the hyperthermia excitation field and the another hyperthermia excitation field are displaced by a quarter cycle to create a rotating hyperthermia excitation field.

9. The system of claim 1 wherein the hyperthermia system is configured to utilize the RF system of the MRI system to generate the hyperthermia excitation field.

10. The system of claim 1 wherein the hyperthermia system includes an RF heating coil system configured to be driven in conjunction with a transmit and receive system of the MRI system by the RF system of the MRI system.

11. A method for performing a magnetic nanoparticle hyperthermia (MPH) process on a subject having received a dose of a magnetic nanoparticle configured to bind to tissue within a target area of the subject and monitoring the MPH process using a magnetic resonance imaging (MRI) system, the method comprising the steps of:
a) acquiring, with the MRI system having a static magnetic field, images of the target area to determine a desired binding of the magnetic nanoparticle to tissue within the target area;
b) producing, with the MRI system, a hyperthermia excitation field configured to cause the magnetic nanoparticle to rotate at a lag with respect to a magnetic field experienced by the magnetic nanoparticle to cause a temperature increase in the target area;
c) acquiring medical imaging data from the target area during the temperature increase in the target area; and
d) reconstructing images of the target area from the medical imaging data to provide real-time feedback indicating at least one of a spatial distribution of the nanoparticle in and a temperature of the target area.

12. The method of claim 11 further comprising selecting at least one of an amplitude, frequency, and phase of the hyperthermia excitation field based on at least one of a radius of the magnetic nanoparticle and a time constant of the magnetic nanoparticle.

13. The method of claim 11 wherein steps b) and c) are repeatedly interleaved such that the hyperthermia excitation field is interleaved with RF excitation pulses configured to acquire the medical imaging data.

14. The method of claim 11 wherein the hyperthermia excitation field is substantially orthogonal to the static magnetic field.

15. The method of claim 14 wherein the hyperthermia excitation field is at least one of rotating and sinusoidal.

16. The method of claim 15 wherein step b) further includes producing another hyperthermia excitation field to create an alternating magnetic field substantially transverse to the static magnetic field of the MRI system.

17. The method of claim 16 wherein step b) includes displacing the hyperthermia excitation field and the another hyperthermia excitation field by a quarter cycle to create the rotating hyperthermia excitation field.
